(19) United States
(12) Patent Application Publication
(10) Pub. No.: US 2008/0199900 A1
(43) Pub. Date: Aug. 21, 2008

(54) Disposable Device For One Or More Introductions, Treatment And Sampling Of Biological Material From At Least One Of The Separation Phases Present Within The Device, Under Sterility Conditions and Constant Pressure

(75) Inventors: Alberto Signore, Rome (IT); Alessio Annovazzi, Rome (IT)

Correspondence Address: YOUNG & THOMPSON, 209 Madison Street, Suite 500, ALEXANDRIA, VA, 22314, US

(73) Assignee: UNIVERSITA' DEGLI STUDI DI ROMA "LA SPIENZA", Roma (IT)

(21) Appl. No.: 11/659,076

(22) PCT Filed: Jul. 7, 2005
(86) PCT No.: PCT/IT05/00390

§ 371 Date: May 1, 2007

(30) Foreign Application Priority Data
Aug. 4, 2004 (IT) RM2004 A 000397

(51) Int. Cl. C12Q 1/02; G01N 33/50

(52) U.S. Cl. 435/29; 422/68.1

(57) ABSTRACT

A disposable device (1) for introduction, treatment and sampling of biological material from at last one of the separation phases under sterility and constant pressure conditions includes a sealed sterile test tube (2), having a first opening (4), or sampling opening, a second opening (7) or inlet opening, and a third opening (8), the first opening (4) providing the passage of a needle (5), coupling between the needle (5) and the first opening (4) being a sealing coupling by an elastic element (6) allowing translation in a substantially vertical direction and inclination of the needle (5). The second opening is sealed by a membrane (7) made of a material allowing the piercing by a syringe needle and closing back after the removal of the needle, the third opening (8) providing a sealing valve (8) balancing the pressure inside the test tube and environmental pressure.

The present invention concerns a disposable device for multiple introductions, treatment and sampling of biological material from one of the separation phases under sterility and constant pressure conditions.

More specifically, the invention concerns a device of the above kind for example allowing to carry out ex-vivo marking of figuration blood elements, such as leucocytes, red cells and platelets, with radioactive isotopes or other marking material; under sterility and safety conditions for the operator.

In the following, the specification will be mainly addressed to the use of the device for carrying out the ex-vivo marking of leucocytes with radioactive isotopes under sterility conditions, but it is well evident that the device according to the invention can be used for each application, such as extraction of nucleic acids and proteins, requiring admission and sampling of substances from a container, under sterility conditions, in two or more phases and by the sampling within the container occurring at different levels.

Thus, referring particularly to the scintigraphy with marked leucocytes, it is known that it is a diagnostic investigation aiming identifying presence of possible infection focus in organism! that cannot be individuated by other techniques.

The above method is based on intravenous injection of patient white cells to which a radio-mimetic agent has been previously legato in vitro, ^{99m}Tc-HMPAO -(Roca et al. 1998) or ¹¹¹In-oxina (Thakur et al. 1977), so as to identify seats wherein they are accumulated putting into evidence the presence of an infection focus.

As it is well known, scintigraphy with labelled leucocytes is used in clinical practice for diagnosis of pathologies characterised by the presence of acute inflammation areas. Scintigraphy with labelled leucocytes is considered very important for diagnosis and follow-up of articular prosthesis (Larikka et al. 2001) and vascular (Liberatore et al. 1998) infections, as well as of osteomyelitis (Krznaric et al. 1996). It further has an important role for intestinal chronic inflammatory diseases (Martin-Comin et al. 1999) and for post-surgical neurological infections (Medina et al. 2000).

At present, preparation of labelled leucocytes occurs by various protocols, all very similar each other.

Mainly used protocol (Roca et al. 1998) provides that:

50 cc of blood are taken from a forearm vein of the patient by a syringe containing a suitable dose of anti-coagulant (ACD);

under sterility conditions, by the use of a laminar flow hood and of a centrifugal machine, white cells are purified by the other blood cells and then labelled by a radioactive substance (^{99m}Tc-HMPAO or ¹¹¹In-oxina or ^{99m}TcSnF); said operation takes about 90 minutes;

at the end of the previous operation, suspension containing white cells is injected in patient by intravenous mode and then images are obtained by a gamma-camera of the whole body or of single body portions, after about 3-4 hours and about 24 hours from the injection.

Different protocols for labelling of white cells employing ^{99m}Tc-HMPAO (Karesh et al. 1897; Solanki et al. 1988; Roca et al. 1998) or ¹¹¹In-oxina (Thakur et al. 1977) are different each other only for minor aspects.

All the known protocols provide the use of a laminar flow hood for carrying out the procedure and skilled technical personnel authorised to handle biological substances potentially infect or cancerogen substances.

Thus, at present, this procedure can be carried out only with nuclear medicine divisions with trained personnel and provided with a laminar flow hood able allowing that the different phases comprising the method can be carried out under sterility and anti-pirogen conditions for patient and safety conditions for operator.

Said conditions are indispensable since labelled white cells are re-injected within the patient at the end of the same procedure and can be carrier of pathogenic micro organism virus.

The above problem remarkably limits diffusion of scintigraphy with labelled leucocytes, notwithstanding it can be a basic test for some pathologies, such as for diagnosis of articular prosthesis and vascular infections and for osteomyelitis.

Impossibility of making the above procedure with a hospital creates noticeable logistic problems for transferring the patients in structures where it is practiced: further consequence of the reduced availability is unavoidably a long waiting list for patients. The other remarkable problem, relevant to the separation and labelling procedure of white cells, is the potential infective risk for exposition of the operator carrying out the procedure for handling the blood.

In view of the above, it is well evident the-advantage of having a device as the one proposed according to the present invention that allows remarkably limiting the infection risk.

Another object of the present invention is that of providing a device allowing the separation and labelling of white cells as one of each commercially available radio-mimetic agent (^{99m}Tc-HMPAO or ¹¹¹In-oxina or others) without the need of having a laminar flow hood, while the standard procedure described by Roca et al. (1998) provides the “open work” labelling but under the laminar flow hood in order to maintain the sterility conditions of the preparation that must be then re-injected within the patient, requiring the use of disposable sterile materials (Pasteur pipette, falcon test tubes, etc.), training of personnel skilled for working under sterility conditions, execution of periodic sterility controls of hood and of the other apparatuses employed, high risk of viral and bacterial contamination of the preparation, and high risk of contamination of the operator when handling infect blood. To the above, it is necessary adding the investment and maintenance expenses for the laminar flow hood.

By the solution according to the present invention, it is on the contrary possible obtaining:
reduction of disposable material to be used;
reduction of the risk of infecting the preparation;
reduction of the infection risk for the operator;
higher execution simplicity of the procedure not requiring specialised personnel;
reduction of quality controls to be carried out.

It is therefore specific object of the present invention a disposable device for one or more introductions, treatment and sampling of biological material from at last one of the separation phases under sterility and constant pressure conditions comprising a sealed sterile test tube, said sterile test tube, comprised of glass or plastic material, providing a first upper opening, or sampling opening, a second opening or inlet opening, and a third opening, or filtered opening for maintaining the sterile atmospheric pressure, said first opening providing the passage of a needle, said needle having a length sufficient to reach the bottom of the test tube, the coupling between said needle and the first opening being a sealing coupling obtained by an elastic element allowing the translation in a substantially vertical direction and further allowing inclination of the needle; said second opening being sealed by a membrane comprised of a material allowing the piercing by a syringe needle and closing back after the removal of the needle; said third opening providing a filter or sealing valve balancing the pressure inside the test tube and the environmental pressure, guaranteeing sterility of the content; said device having dimensions and materials allowing its centrifugal.

Preferably, according to the invention; said test tube can be provided with a plug coupable with the same test tube by a sealing coupling.

Always according to the invention, said plug can be integral with the same test tube.

Still according to the invention, coupling between said first opening and said movable needle can be comprised of a bellow or of an elastic sheath.

Furthermore, according to the invention, said membrane is integral with said test tube or with the plug of said test tube.

Always according to the invention, said third opening can be provided with a filter, in order to prevent penetration of bacteria within the test tube.

The invention further concerns a method for labelling of figuration elements of blood, particularly leucocytes, with radioactive isotopes under sterility conditions employing a device as described in the above and providing the following steps:
sampling by a syringe containing anticoagulant agent and sedimentation agent, an amount of blood using a butterfly device and gently mixing the contents;
leaving sedimentation the blood within the syringe for 30-60 minutes;
at the end of sedimentation, transferring the plasma rich of cells from the syringe to the device, introducing the butterfly needle within the second opening;
centrifugating the device for 5 minutes, creating on the bottom of the test tube a red coloured pellet containing the leucocytes, and then introducing a disposable sterile syringe within the needle introduced within the first opening and removing the supernatant from the device;
after having suspended again the cellular pellet gently agitating the device, adding ^{99m}Tc-HMPAO, already prepared, by a disposable sterile syringe through the second opening of the device;
at the end of the incubation time necessary (about 10 minutes), adding physiological sterile solution by disposable sterile syringe through the first opening and centrifugating for 5 minutes;
introducing a disposable sterile syringe in the needle introduced in the first opening and removing the supernatant from the device;
adding 2-3 ml of physiological sterile solution by disposable sterile syringe through the second opening and gently suspending again the pellet before taking again the labelled cells to be injected in the patient, introducing a disposable sterile syringe in the needle of the first opening and sampling all the contents of the device.

The present invention will be now described, for illustrative but not limitative purposes, according to its preferred embodiments, with particular reference to the figures of the enclosed drawings, wherein:

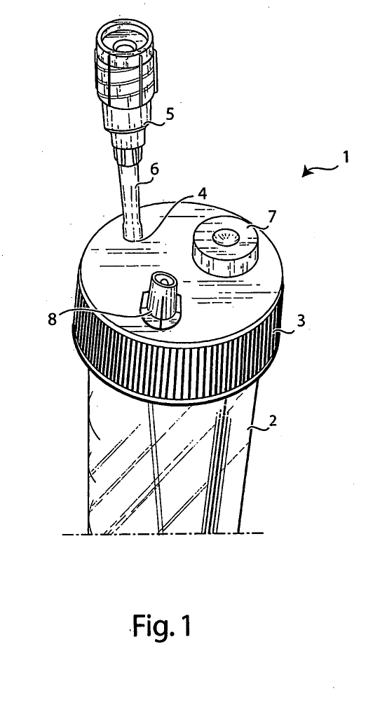

FIG. 1 is a perspective view of an embodiment of a device according to the invention;

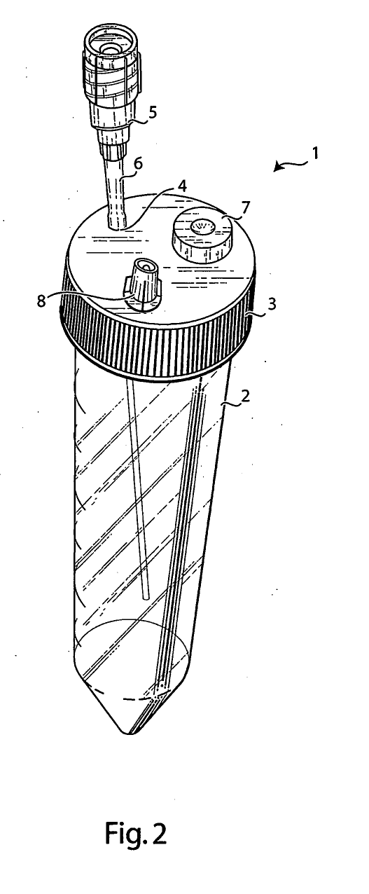

FIG. 2 is a perspective view of a particular of the device of FIG. 1; and

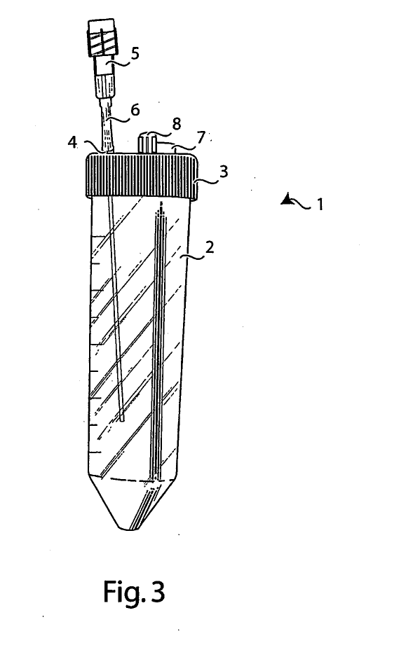

FIG. 3 is a front view of the device of FIG. 1.

In the figures it is shown a prototype of a disposable device according to the invention particularly realised for ex vivo labelling of leucocytes with radioactive isotopes under sterility conditions. However, it must be once more evidenced that, simply modifying material and dimensions of the device, it will be possible realising the same for a specific different application.

Observing now specifically FIGS. 1-3, it is shown a disposable device, generically indicated by reference number 1, providing a sterile test tube, e.g. a test tube with a volume of 60 ml and a length of 12 cm, with a plug 3, that can be integral with the test tube 2 or sealingly coupled with the same, provided with three openings, that will be described in greater detail in the following.

Said test tube 2 provides a conical narrowing on its bottom for collection of leucocytes pellet after centrifugations.

A needle 5 is provided in correspondence of the first opening 4, for example a needle having a diameter 19 G and long 12 cm that can be handled outside the test tube 2.

In correspondence of the coupling between needle 5 and opening 4 a bellow or sheath 6 is provided, sealing coupled both to the plug 3 of the test tube and to the needle 5, allowing mobility of needle 5 e.g. for 2 cm within the test tube 2, ensuring its sterility. In other words, opening 4 is the outlet of the device according to the invention, through which it is possible sampling contents of device 1 at different heights along the test tube 2 thanks to the action of the bellow 6.

Projection of needle 5 and sheath 6 from the plug 3 of test tube 2 must be sufficient not to hinder the centrifugation phase.

Second opening 7 is comprised of a membrane, integrally coupled with the plug 3, and thus not removable, said membrane being comprised of a material having features allowing its disinfection with alcohol and its piercing, even more times, by a needle.

Material of membrane 7 must re-close on itself after the piercing by the needle and the removal of the same needle, so as to maintain sterility of test tube 2.

Said opening 7 is used for introduction of materials inside the test tube 2 by a needle coupled with a suitable syringe (not shown).

Third opening 8 provides a filter, for example a 0.2μ filter, guaranteeing the atmospheric pressure within the test tube 2, and at the same time preventing penetration of bacteria within the same test tube.

As already said, device 1 according to the invention is a disposable device, and it is realised in such a way to be introduced in every kind of centrifugal machine, since needle 5 is flexible and adaptable to different rotors.

Coming now to describe, for exemplificative and not limitative purposes, the steps of the use of device 1 according to the invention for ex-vivo labelling of leucocytes with radioactive leucocytes, under sterility conditions, the following sequence will be followed:
by a 60 ml syringe containing 7.5 ml of anticoagulant agent (ACD) and 7.5 ml of sedimentation agent (HES), 45 ml of blood are sampled using a 19 G butterfly device and gently mixing the contents;
leaving sedimentation the blood within the syringe for 30-60 minutes;
at the end of sedimentation, transferring the plasma rich of cells from the syringe to the device, introducing the butterfly needle within the opening 7;
centrifugating the device for 5 minutes at 150 g. On the bottom of the test tube 2 a red coloured pellet containing the leucocytes will be created. Then a 20 cc disposable sterile syringe is introduced within the needle introduced within the opening 5 and the supernatant is removed from the device;
after having suspended again the cellular pellet gently agitating the device, adding ^{99m}Tc-HMPAO, already prepared, by a disposable sterile syringe through the opening 7 of the device;
at the end of the incubation time necessary (about 10 minutes), adding physiological sterile solution by disposable sterile syringe through the opening 7 and centrifugating for 5 minutes at 150 g;
introducing a 10 cc disposable sterile syringe in the needle 5 introduced in the opening 4 and removing the supernatant from the device;
adding 2-3 ml of physiological sterile solution by disposable sterile syringe through the opening 7 and gently suspending again the pellet before taking again the labelled cells to be injected in the patient. Also the last operation is carried out introducing a 5 cc disposable sterile syringe in the needle 5 of the opening 4 and sampling all the contents of the device.

Summarising, the whole cellular labelling operation requires four piercings of membrane 7 and three connections of a sterile syringe with needle 5. The whole operation requires only seven disposable sterile syringes, beside the device 1 according to the invention, thus obtaining a remarkable save of disposable sterile material with respect to the standard methods (Roca et al. 1998), but mainly difficulties are remarkably reduced for the operator and consequently -the possibilities of bacterial contamination of preparation are strongly reduced.

The present invention has been described for illustrative but not limitative purposes, according to its preferred embodiments, but it is to be understood that modifications and/or changes can be introduced by those skilled in the art without departing from the relevant scope as defined in the enclosed claims.

1. Disposable device for one or more introductions, treatment and sampling of biological material from at last one of the separation phases under sterility and constant pressure conditions comprising a sealed sterile test tube, said sterile test tube, comprised of glass or plastic material, providing a first upper opening, or sampling opening, a second opening or inlet opening, and a third opening, or filtered opening for maintaining the sterile atmospheric pressure, said first opening providing the passage of a needle, said needle having a length sufficient to reach the bottom of the test tube, the coupling between said needle and the first opening being a sealing coupling obtained by an elastic element allowing the translation in a substantially vertical direction and further allowing inclination of the needle; said second opening being sealed by a membrane comprised of a material allowing the piercing by a syringe needle and closing back after the removal of the needle; said third opening providing a filter or sealing valve balancing the pressure inside the test tube and the environmental pressure, guaranteeing sterility of the content; said device having dimensions and materials allowing its centrifugal.

2. Device according to claim 1, characterised in that said test tube is provided with a plug coupable with the same test tube by a sealing coupling.

3. Device according to claim 1, characterised in that said plug is integral with the same test tube.

4. Device according to claim 1, characterised in that coupling between said first opening and said movable needle is comprised of a bellow or of an elastic sheath.

5. Device according to claim 1, characterised in that said membrane is integral with said test tube or with the plug of said test tube.

6. Device according to claim 1, characterised in that said third opening is provided with a filter, in order to prevent penetration of bacteria within the test tube.

7. Method for labelling of figuration elements of blood, particularly leucocytes, with radioactive isotopes under sterility conditions employing a device according to claim 1 characterised in that it provides the following steps:
sampling by a syringe containing anticoagulant agent and sedimentation agent, an amount of blood using a butterfly device and gently mixing the contents;
leaving sedimentation the blood within the syringe for 30-60 minutes;
at the end of sedimentation, transferring the plasma rich of cells from the syringe to the device, introducing the butterfly needle within the second opening;
centrifugating the device for 5 minutes, creating on the bottom of the test tube a red coloured pellet containing the leucocytes, and then introducing a disposable sterile syringe within the needle introduced within the first opening and removing the supernatant from the device;
after having suspended again the cellular pellet gently agitating the device, adding ^{99m}Tc-HMPAO, already prepared, by a disposable sterile syringe through the second opening of the device;
at the end of the incubation time necessary (about 10 minutes), adding physiological sterile solution by disposable sterile syringe through the first opening and centrifugating for 5 minutes;
introducing a disposable sterile syringe in the needle introduced in the first opening and removing the supernatant from the device;
adding 2-3 ml of physiological sterile solution by disposable sterile syringe through the second opening and gently suspending again the pellet before taking again the labelled cells to be injected in the patient, introducing a disposable sterile syringe in the needle of the first opening and sampling all the contents of the device.
